(19) United States
(12) Patent Application Publication
(10) Pub. No.: US 2022/0000748 A1
(43) Pub. Date: Jan. 6, 2022

(54) BIS(TRIETHOXYSILYLPROPYL)AMINES IN COMBINATION WITH A THICKENING AGENT

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/290,725

(22) PCT Filed: Oct. 31, 2019
(86) PCT No.: PCT/EP2019/079778

§ 371 Date: Apr. 30, 2021

(30) Foreign Application Priority Data
Oct. 31, 2018 (DE) 10 2018 127 194.8

(51) Int. Cl. A61K 8/58; A61K 8/73; A61K 8/44; A61Q 5/12

(52) U.S. Cl. CPC A61K 8/585; A61K 8/733; A61K 2800/5922; A61Q 5/12; A61K 2800/48; A61K 8/44

(57) ABSTRACT

The present disclosure relates to an active ingredient composition for shape change and surface modification of human hair. In particular, the present disclosure relates to a cosmetic agent for treating a keratinous material comprising a) at least one organic silicon compound and b) at least one thickener, the cosmetic agent being particularly suitable for treating colored hair.

### CROSS-REFERENCE TO RELATED APPLICATION

This application is a U.S. National-Stage entry under 35 U.S.C. § 371 based on International Application No. PCT/EP2019/079778, filed Oct. 31, 2019, which was published under PCT Article 21(2) and which claims priority to German Application No. 10 2018 127 194.8, filed Oct. 31, 2018, which are all hereby incorporated in their entirety by reference.

### TECHNICAL FIELD

The present disclosure relates to cosmetic agents for treating a keratin material, wherein the agent comprises an organic silicon compound and at least one thickener, and to the use of the cosmetic agent.

### BACKGROUND

The exposure of hair to external chemicals from a variety of different sources poses challenges for the development of cosmetic care products. Air and water impurities have a detrimental effect on skin and hair. The most important air pollutants include polycyclic aromatic hydrocarbons, volatile organic compounds, nitrogen oxides (NOx), particles and cigarette smoke. The action of various atmospheric pollutants can be intensified in the presence of other atmospheric pollutants and under UV radiation action.

It is known that the toxicity of gaseous pollutants of air, such as sulfur dioxide, ozone, and nitrogen oxides, is related in particular to their initiator activity for free radicals which cause damage to living organisms. Free radicals are metabolites that also occur naturally in the body. In large quantities, free radicals can promote irritation and inflammation and accelerate the process of aging. In this case, one speaks of “oxidative damage” Free radicals can also cause visible hair damage, for example, as a reduction in shine as well as grip and/or fading of hair color.

Furthermore, often changing consumer demands for a certain hair texture are associated with recurring hair chemical stress. For example, hair colorings stress the hair, as a result of which special, intensive care may be necessary.

In the prior art, organosilicon compounds from the group of silanes comprising at least one hydroxy group and/or hydrolyzable group are described. Due to the presence of hydroxy groups and/or hydrolyzable groups, silanes are reactive substances that hydrolyze or oligomerize or polymerize in the presence of water. The oligomerization or polymerization of the silanes initiated by the presence of the water, when applied to a keratinous material, ultimately leads to the formation of a film that can provide a protective effect.

### BRIEF SUMMARY

A cosmetic agent for the treatment of a keratinous material is provided herein. The cosmetic agent includes

at least one organic silicon compound and

at least one thickener.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The aim of the present disclosure is to provide a cosmetic product which enables the hair to have a good care effect, improves structural protection against chemical stress and at the same time represents a product that is easy to handle in terms of its physical properties.

This problem is solved by a cosmetic agent for the treatment of a keratinous material, comprising

a) at least one organic silicon compound and
b) at least one thickener.

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. In particular, keratinous material is understood to mean human hair, especially head and/or beard hair.

As a first ingredient essential to the invention, the cosmetic agent for treating a keratinous material contains at least one organic silicon compound. Preferred organic silicon compounds are selected from silanes having one, two or three silicon atoms, wherein the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolyzable groups per molecule.

In a particularly preferred embodiment, the agent for treating a keratinous material comprises at least one organic silicon compound selected from silanes having one, two or three silicon atoms, the organic silicon compound further comprising one or more basic groups and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a Di(C₁-C₆)alkylamino group.

The hydrolyzable group (s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH₂—CH₃. The residues R′, R′ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),
where

R₁, R₂ both represent a hydrogen atom, and

L represents a linear, divalent C₁-C₆-alkylene group, preferably a propylene group (—CH₂—CH₂—CH₂—) or an ethylene group (—CH₂—CH₂—),

R₃, R₄ independently represent a methyl group or an ethyl group, and

a stands for the number 3 and
b stands for the number 0.

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where

R5, R5′, R5″ independently represent a hydrogen atom or a C₁-C₆ alkyl group,

R6, R6′ and R6″ independently represent a C₁-C₆ alkyl group,

A, A′, A″, A′″ and A″″ independently represent a linear or branched divalent C₁-C₂₀ alkylene group,

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d″}(OR₅″)_{c}″ (III),

c, stands for an integer from 1 to 3,

d stands for the integer 3−c,

c′ stands for an integer from 1 to 3,

d′ stands for the integer 3−c′,

c″ stands for an integer from 1 to 3,

d″ stands for the integer 3−c″,

e stands for 0 or 1,

f stands for 0 or 1,

g stands for 0 or 1,

h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L-, which stands for a linear or branched, divalent C₁-C₂₀ alkylene group.

Preferably, -L- represents a linear, divalent C₁-C₂₀ alkylene group. More preferably -L- stands for a linear divalent C₁-C₆ alkylene group. Particularly preferred -L stands for a methylene group (—CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R4)_{b}, R₃ is hydrogen or C₁-C₆ alkyl group, and R4 is C₁-C₆ alkyl group. In particular, R₃ and R₄ independently of each other represent a methyl group or an ethyl group.

Here a stands for an integer from 1 to 3, and b stands for the integer 3−a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are

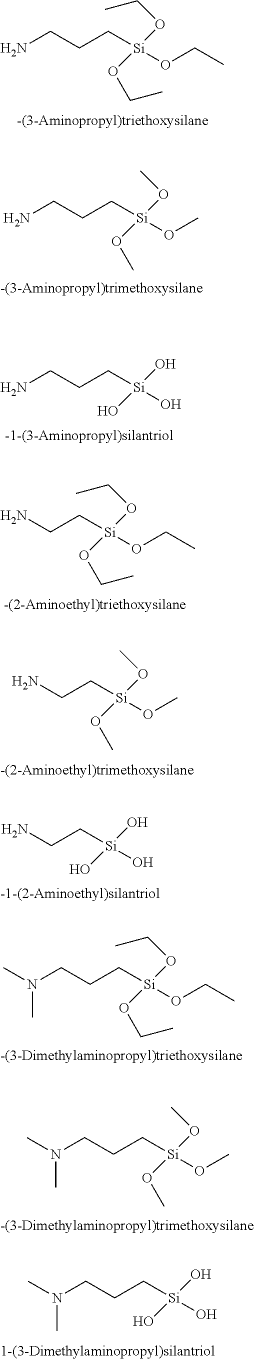

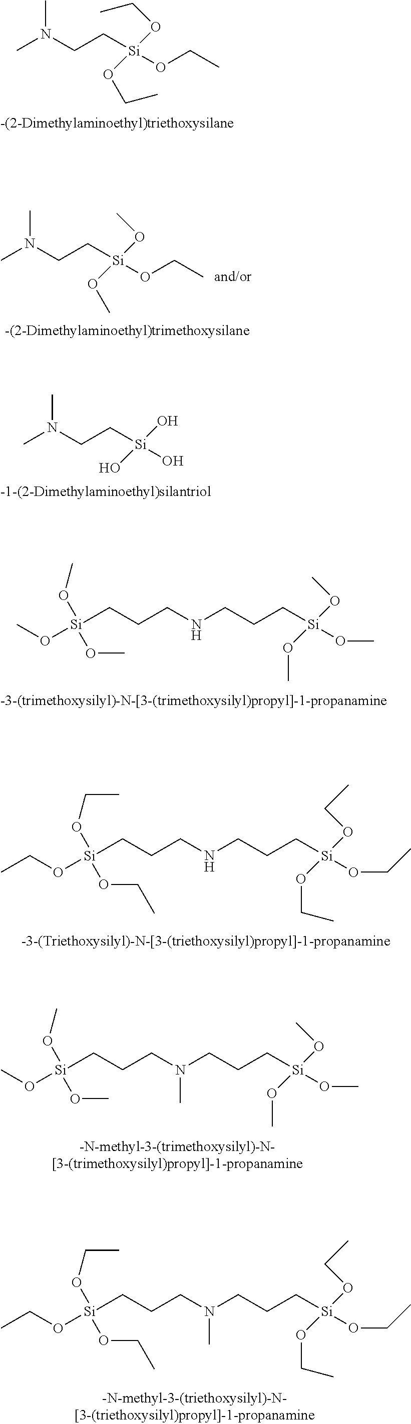

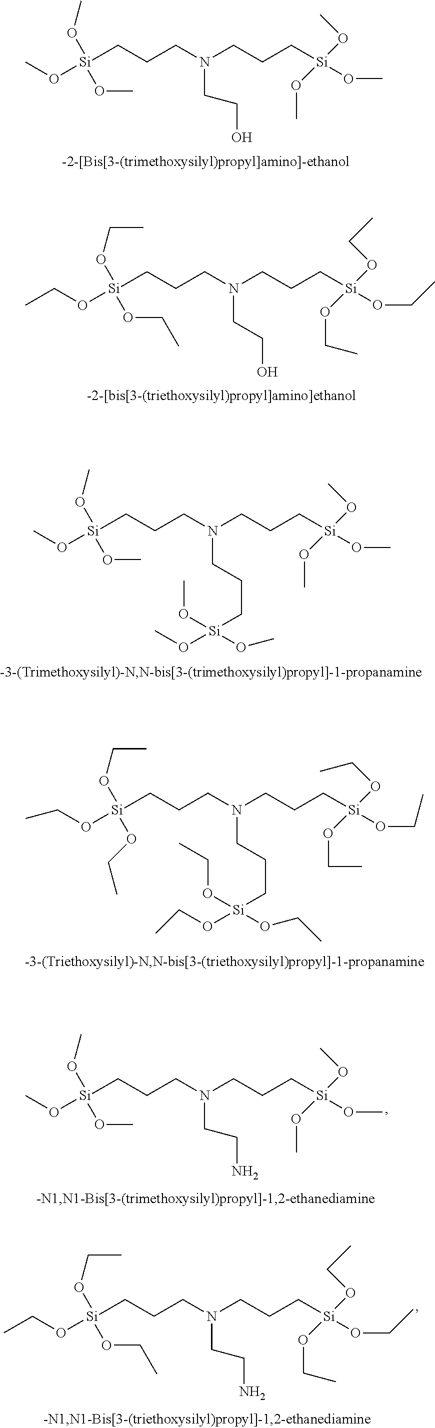

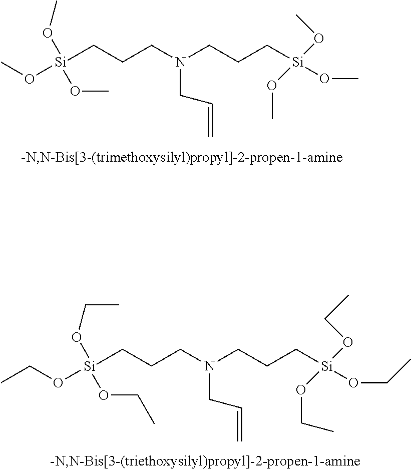

The aforementioned organic silicon compound of formula (I) is commercially available.

(3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

The organosilicon compounds of formula (II) as contemplated herein each carry the silicon-containing groups (R₅O)_{c}(R₆)_{d}Si— and —Si(R₆′)_{d′}(OR₅′)_{d} at both ends.

In the central part of the molecule of formula (II) there are the groups -(A)_{c}- and —[NR₇-(A′)]_{f}-

and —[O-(A″)]_{g}- and —[NR₈-(A′″)]ₕ—Here, each of the radicals e, f, g and h can independently of one another stand for the number 0 or 1, with the proviso that at least one of the radicals e, f, g and h is different from 0. In other words, an organic silicon compound of formula (II) as contemplated herein contains at least one grouping from the group of -(A)- and [NR₇-(A′)]- and —[O-(A″)]- and [NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3−c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3−c′. If c′ stands for the number 3, then d′ is 0. If c′ stands for the number 2, then d′ is 1. If c′ stands for the number 1, then d′ is 2.

A very high anti-pollution effect of the agent for the treatment of a keratin material could be obtained if the residues c and c′ both represent the number 3. In this case d and d′ both stand for the number 0.

In a further preferred embodiment, the agent for treating a keratin material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where

R5 and R5′ independently represent a methyl group or an ethyl group,

c and c′ both stand for the number 3 and

d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}-[O-(A″)]_{g}[NR₈-(A′″)]ₕ-Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and —[NR7-(A′)]_{f}- and —[O-(A″)]_{g}- and [NR8-(A′″)]ₕ- are located in the middle part of the organic silicon compound of formula (II).

In this context, the presence of certain groups has proved to be particularly advantageous in terms of increasing the “anti-pollution” effect. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping [NR₇-(A′)]-.

When the radical h represents the number 1, the organic silicon compound of formula (II) contains a structural grouping [NR₈-(A″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III),

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound as contemplated herein contains the grouping [NR₇-(A′)] but not the grouping —[NR₈-(A′″)]. If the residue R7 is a grouping of formula (III), the agent for treating a keratin material contains an organic silicon compound with 3 reactive silane groups.

In a further preferred embodiment, the agent for treating a keratin material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where

e and f both stand for the number 1,

g and h both stand for the number 0,

A and A′ independently of one another represent a linear, divalent C₁-C₆ alkylene group and

R7 represents a hydrogen atom, a methyl group, a 2-hydroxyethyl group, a 2-alkenyl group, a 2-aminoethyl group or a group of formula (III).

In another preferred embodiment, the agent for treating a keratin material contains

at least one organic silicon compound of formula (II), wherein

e and f both stand for the number 1,

g and h both stand for the number 0,

A and A′ independently of one another represent a methylene group (—CH₂—), an ethylene group (—CH₂—CH₂—) or a propylene group (—CH₂—CH₂—CH₂),

and

R7 represents a hydrogen atom, a methyl group, a 2-hydroxyethyl group, a 2-alkenyl group, a 2-aminoethyl group or a group of formula (III).

Organic silicon compounds of formula (II) that are suitable for solving the problem are

The aforementioned organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, having the CAS number 13497-18-2, can be purchased, for example, from Sigma-Aldrich or is commercially available under the product name Dynasylan 1122 from Evonik.

N-methyl-3-(trimethoxysilyl)-N-[3-(trimethoxysilyl)propyl]1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous if the agent for treating a keratinous material applied to the hair contains at least one organic silicon compound of formula (IV)

R₉Si(OR₁₀)ₖ(R₁₁)ₘ (IV),

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV).

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3−k.

In a further preferred embodiment, the agent for treating a keratin material contains, in addition to the organic silicon compounds of formula (I), at least one further organic silicon compound of formula (IV)

R₉Si(OR₁₀)ₖ(R₁₁)ₘ (IV).

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3−k.

In a likewise preferred embodiment, the agent for treating a keratin material contains at least one further organic silicon compound of formula (IV) in addition to the organic silicon compounds of formula (II)

R₉Si(OR₁₀)ₖ(R₁₁)ₘ (IV).

where

R₉ represents a C₁-C₁₂, alkyl group,

R₁₀ represents a hydrogen atom or a C₁-C₆ alkyl group,

R₁₁ represents a C₁-C₆ alkyl group

k is an integer from 1 to 3, and

m stands for the integer 3−k.

In a further preferred embodiment, the agent for treating a keratin material contains, in addition to the organic silicon compounds of formula (I) and (II), at least one further organic silicon compound of formula (IV)

R₉Si(OR₁₀)ₖ(R₁₁)ₘ (IV).

where

R₉ represents a C₁-C₁₂, alkyl group,

R₁₀ represents a hydrogen atom or a C₁-C₆ alkyl group,

R₁₁ represents a C₁-C₆ alkyl group

k is an integer from 1 to 3, and

m stands for the integer 3−k.

In the organic silicon compounds of formula (IV), the radical R₉ represents a C₁-C₁₂, alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. It is particularly preferred that R₁₀ represents a methyl group or an ethyl group.

In the organic silicon compounds of formula (IV), the radical R₁₁ represents a C₁-C₆ alkyl group. It is particularly preferred that R₁₁ represents a methyl group or an ethyl group.

Furthermore k stands for a whole number from 1 to 3, and m stands for the whole number 3−k. If k stands for the number 3, then m is equal to 0. If k stands for the number 2, then m is equal to 1. If k stands for the number 1, then m is equal to 2.

A very high “anti-pollution” effect could be obtained if the agent for treating a keratinous material contains at least one organic silicon compound of formula (IV) in which the radical k represents the number 3. In this case the rest m stands for the number 0.

Organic silicon compounds of the formula (IV) that are particularly suitable for solving the problem are

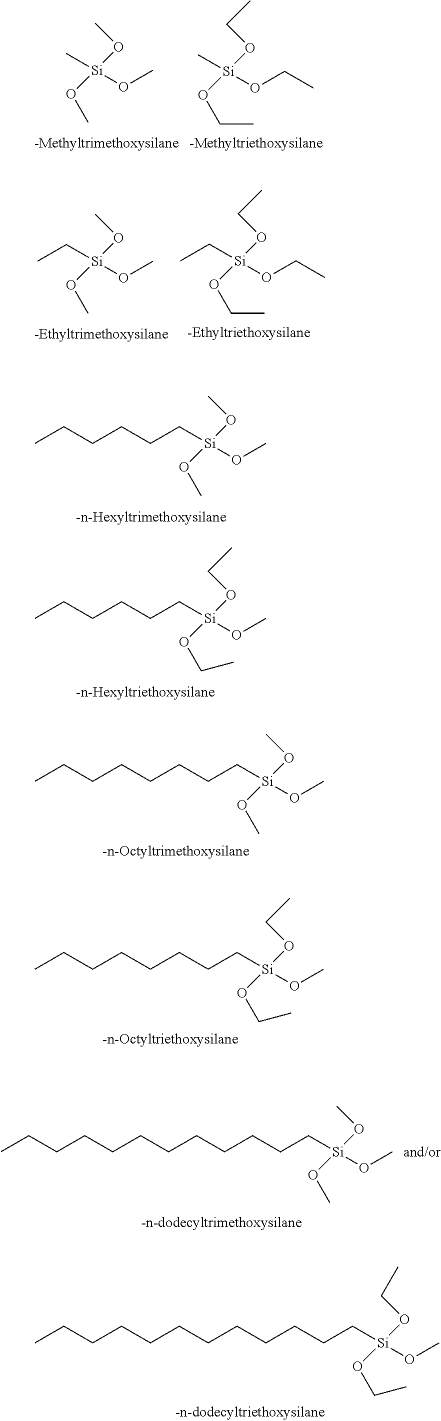

and also propyltrimethoxysilane, propyltriethoxysilane, octadecyltrimethoxysilane and/or octadecyltriethoxysilane.

The organic silicon compounds described above are reactive compounds.

In this context, it has turned out to be particularly preferred if the agent contains triethoxysilane as an organic silicon compound (3-aminopropyl).

It was found that particularly stable and uniform films could be obtained on the keratinous material even when the agent contained two structurally different organic silicon compounds.

In a preferred embodiment, an agent exemplified in that it comprises at least one organic silicone compound of formula (I) and at least one organic silicone compound of formula (IV).

In an explicitly very particularly preferred embodiment, an agent is exemplified in that it contains at least one organic silicone compound of formula (I) selected from the group of (3-aminopropyl)triethoxysilane and (3-aminopropyl)trimethoxysilane, and additionally containing at least one organic silicone compound of formula (IV) selected from the group of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an agent is exemplified in that the agent—contains, based on the total weight of the agent—:

from about 0.5 to about 5 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and

From about 3.2 to about 10 wt. % of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in the case of organic silicon compounds having at least one hydrolyzable group. The hydrolysis products and/or organic silicon compounds having at least one hydroxyl group can react with one another in a condensation reaction. For this reason, both the organosilicon compounds having at least one hydrolyzable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium of monomeric organic silicon compounds shifts to condensation product.

In the context of the present invention, figures in wt.-% are—unless otherwise stated—always based on the total weight of the cosmetic agent.

As a second ingredient essential to the invention, the cosmetic agent for treating a keratinous material comprises at least one thickener. In the context of the present invention, a thickener means a substance that increases the dynamic viscosity, measured in an SI unit, by more than 10%.

A wide range of substance classes can be considered as thickeners. Known systems include salt thickening of a surfactant system, thickening by nonionic, anionic, amphoteric, or cationic polymers.

According to preferred embodiments of the present invention, the thickener comprises a crosslinked polymer, a polyelectrolyte or a crosslinked polyelectrolyte. Crosslinked polymers (and crosslinked polyelectrolytes) should be understood to mean polymers that are chemically crosslinked, that are physically crosslinked by association of polymer sections in a fluid medium, or that are formed by polymerization of monomers with two or more polymerizable functionalities.

According to preferred embodiments, the thickener is selected from the group of hydroxyethyl cellulose, carboxymethyl celluloses, sodium carboxymethyl cellulose, hydroxypropyl methyl cellulose, hydroxypropyl cellulose, ethyl hydroxyethyl cellulose, ethyl cellulose, methyl ethyl cellulose, methyl cellulose, cellulose powder, microcrystalline cellulose, polyvinyl alcohols, polyacrylic acid and polymethacrylic acids, polyacrylamides, polyvinylpyrrolidone, polyethylene glycols, alginic acid, sodium alginate, potassium alginate, ammonium alginate, calcium alginate, propylene glycol alginate, agar-agar, carrageenan, furcellaran, locust bean gum, guar gum, tragacanth, gum arabic, xanthan gum, chitosan, cationized honey, karaya, Indian tragacanth, tara gum, locust bean gum, gellan gum, pectin and amidated pectin, especially xanthan gum.

In the course of the work leading to the present invention, it has been found that, in order to achieve advantageous properties in terms of manageability while at the same time protecting the structure, it is advantageous if the organic silicon compounds, in particular 3-(triethoxysilyl)-N-[3-(triethoxysilyl)propyl]-1-propanamine, i.e. a bis(triethoxysilylpropyl)amine, and/or (3-aminopropyl)triethoxysilane, i.e. an aminopropyltriethoxysilane (AMEO), is combined with a thickener.

Particularly preferred thickeners include alginic acid and/or salts thereof, such as sodium alginate, potassium alginate, calcium alginate and/or ammonium alginate.

According to a preferred embodiment of the present invention, the amount of thickener in the cosmetic composition is from about 0.001 to about 5% by weight, preferably from about 0.005 to about 2% by weight, more preferably from about 0.01 to about 1% by weight, most preferably from about 0.05 to about 0.5% by weight, based on the total weight of the cosmetic composition.

In the following, further ingredients of the cosmetic product are described, which may be contained in the products in addition to the previously described mandatory and optional ingredients.

It may be preferred that the agent for treating a keratinous material further comprises from about 0.001 to about 20% by weight of at least one quaternary compound. This applies in particular to agents which additionally give care properties to the keratin material.

It is preferred that the at least one quaternary compound is selected from at least one of the groups of

i) the monoalkylquats and/or
ii) the esterquats and/or
iii) the quaternary imidazolines of formula (Tkat2),

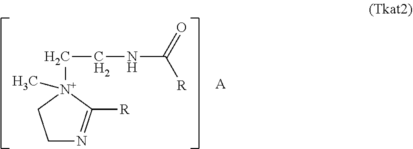

in which the radicals R independently of one another each represent a saturated or unsaturated, linear or branched hydrocarbon radical having a chain length of from about 8 to about 30 carbon atoms and A represents a physiologically tolerated anion, and/or
iv) of the amidoamines and/or cationized amidoamines and/or
v) Poly(methacryloyloxyethyltrimethylammonium compounds) and/or;
vi) quaternized cellulose derivatives, in particular polyquaternium 10, polyquaternium-24, polyquaternium-27, polyquaternium-67, polyquaternium-72, and/or
vii) cationic alkyl polyglycosides and/or
viii) polymeric dimethyldiallylammonium salts and copolymers thereof with esters and amides of acrylic acid and methacrylic acid, in particular polyquaternium-7 and/or
ix) copolymers of vinylpyrrolidone with quaternized derivatives of dialkylaminoalkyl acrylate and methacrylate, especially polyquaternium-11 and/or
x) vinylpyrrolidone-vinylimidazolium methochloride copolymers, in particular polyquaternium-16 and/or
xi) quaternized polyvinyl alcohol and/or
xii) polyquaternium-74
and mixtures thereof.
Furthermore, strengthening compounds, preferably selected from the group of waxes and/or other synthetic polymers, can be contained in the cosmetic product. In other words, waxes and/or other synthetic polymers can be used as strengthening compounds. Ideally, the polysaccharides and the other strengthening compounds result in a film when applied to the keratin material, which on the one hand gives the hairstyle a strong hold, but on the other hand is sufficiently flexible not to break under stress.

For example, polycarboxylic acids can be used in the cosmetic agent as film-forming agents. These are deposited on the hair and establish a temporary deformability of the hair. The film former can be a homopolymer or copolymer derived from itaconic acid. If the film former is present exclusively in the form of polymerized itaconic acid and/or a salt of itaconic acid, the film former forms a homopolymer. An example of such a polymer is PVP/VA/Itaconic Acid Copolymer (INCI).

In addition, an octylacrylamide/acrylates/butylaminoethyl methacrylate copolymer (INCI) may be contained as a stabilizing polymer in the cosmetic product. This copolymer is marketed under the terms “Amphomer®”, Amphomer® HC”, Amphomer® 4961”, Amphomer® EDGE and Amphomer® LV 71” in various variations by Akzo Nobel.

Furthermore, polysaccharides can be contained in the cosmetic products in preferred embodiments. The polysaccharides are used as film formers. As such, they are in addition to the thickener contained in the cosmetic product. According to some embodiments, the polysaccharides preferably present are starch, thermally and/or mechanically treated starch, oxidatively, hydrolytically or oxidized enzymatically degraded starches, oxidized hydrolytically or oxidized enzymatically degraded starches, and chemically modified starches. In this context, all starches are suitable in principle. According to some embodiments, the starch may be, for example, without limitation, starch from corn, wheat, rice, peas, barley, rye, cassava, tapioca, sweet potato, or potato. In some embodiments, native starch is preferably used. Native starch is starch as it is accessible from natural sources, such as those mentioned above. Native starch is a commercially available product and therefore easily obtainable.

According to one embodiment, the polysaccharide is corn, wheat, rice, pea, barley, rye, cassava, tapioca, sweet potato, or potato starch. According to one embodiment, the polysaccharide is the corresponding native starch of the above type. Mixtures of the aforementioned are also suitable. According to one embodiment, the polysaccharide is preferably corn starch, in particular native corn starch. The most preferred is Collamyl 8412 corn starch from Agrabne Wien.

In order to meet the different requirements for agents for the treatment of a keratinous material in the form of an agent for the temporary reshaping of a keratinous material (=styling agent), a large number of synthetic polymers have already been developed as strengthening compounds which can be used in an agent for the treatment of a keratinous material.

The agent for treating a keratinous material may comprise an agent for temporarily reshaping a keratinous material, an agent for cleaning a keratinous material, an agent for caring for a keratinous material, and/or an agent for caring for and cleaning a keratinous material.

According to further preferred embodiments of the present invention, the cosmetic composition comprises at least one cationic surfactant of formula (V),

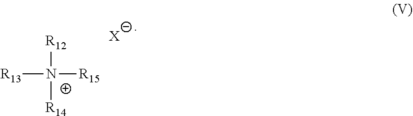

wherein
R₁₂, R₁₃, R₁₄ independently represent a C1-C6 alkyl group, a C2-C6 alkenyl group or a C2-C6 hydroxyalkyl group,
R₁₅ represents a C8-C28 alkyl group, preferably a C10-C22 alkyl group, and
X− stands for a physiologically compatible anion,
and/or the cosmetic composition comprises at least one cationic surfactant of the formula (VI),

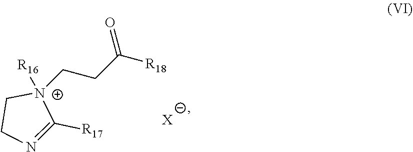

wherein
R16 stands for a C₁-C₆ alkyl group
R₁₇, R₁₈ are independently a C7-C27 alkyl group, preferably a C10-C22 alkyl group, and
X− stands for a physiologically compatible anion,
and/or the cosmetic composition contains at least one cationic surfactant of the formula (VII),

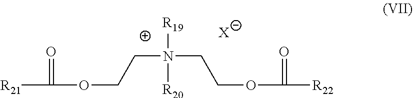

wherein
R₁₉, R₂₀ independently represent a C1-C6 alkyl group, a C2-C6 alkenyl group or a C2-C6 hydroxyalkyl group,
R₂₁, R₂₂ are independently a C7-C27 alkyl group, preferably a C10-C22 alkyl group, and
X− stands for a physiologically compatible anion,
and/or the cosmetic composition contains at least one cationic surfactant of the formula (VIII),

NR₂₃R₂₄R₂₅ (VIII)

wherein
R₂₃, R₂₄ independently represent a C1-C6 alkyl group, a C2-C6 alkenyl group or a C2-C6 hydroxyalkyl group, and
R₂₅ represents a C8-C28 alkyl group, preferably a C10-C22 alkyl group.

The cationic surfactants of formula (VIII) are amine derivatives, so-called pseudoquats. The organic radicals R₂₃, R₂₄ and R₂₅ are bonded directly to the nitrogen atom. In the acidic pH range, these are cationized, i.e. the nitrogen atom is then protonated. The physiologically compatible counterions are suitable as counterions. Stearamidopropyl dimethylamine is particularly preferred among the cationic surfactants of formula (VIII).

According to a preferred embodiment of the present invention, the amount of cationic surfactant is about 0.1 to about 30% by weight, preferably from about 0.5 to about 20% by weight, more preferably from about 1 to about 10% by weight, based on the total weight of the cosmetic composition.

According to a preferred embodiment of the present invention, the cationic surfactant comprises a hydrophobic head group with a cationic charge and one or two hydrophobic end portions, wherein the hydrophobic end portion(s) represent straight-chain or branched, saturated or mono- or polyunsaturated alkyl groups, preferably having a chain length of C6 to C30, more preferably C8 to C26, particularly preferably C10 to C22. According to another preferred embodiment, the cationic surfactant has an ester function, an ether function, a ketone function, an alcohol function, or an amide function.

According to a preferred embodiment of the present invention, the cosmetic agent further comprises a nonionic surfactant. This preferably comprises a nonionic surfactant selected from the group of

Alkyl glucamide comprising a saturated or unsaturated, branched or unbranched C₆ to C₂₂, preferably C₁₀ to C₁₈, more preferably C₁₂ to C₁₆ alkyl group,

An alkyl fructoside comprising a saturated or unsaturated, branched or unbranched C₆ to C₂₂, preferably C₁₀ to C₁₈, more preferably C₁₂ to C₁₆ alkyl group,

alkyl glucoside comprising a saturated or unsaturated, branched or unbranched C₆ to C₂₂, preferably C₁₀ to C₁₈, more preferably C₁₂ to C₁₆ alkyl group, and

Alkyl alcohol alkoxylate of the formula R₁₀(OR₁₁)ₘOH, in which R₁₀ represents a linear or branched C₆ to C₂₂, preferably C₁₀ to C₁₈, more preferably C₁₂ to C₁₆ alkyl group, R₁₁ represents a C₂ to C₄, preferably a C₂ alkyl group, and ₘ represents about 1 to about 10, preferably about 2 to about 6, more preferably 2 to 6.

According to preferred embodiments of the present invention, one or more anionic surfactants are included in the cosmetic agent, which is preferably selected from the group of

straight-chain or branched, saturated or mono- or polyunsaturated alkylsulfonates having from about 8 to about 24, preferably from about 12 to about 22, more preferably from about 16 to about 18, carbon atoms,

linear alpha-olefin sulfonates having from about 8 to about 24, preferably from about 12 to about 22, more preferably from about 16 to about 18, carbon atoms,

Alkyl sulfates and alkyl polyglycol ether sulfates of the formula R₉—O—(CH₂—CH₂O)ₙ—SO₃X, in which R₉ is preferably a straight-chain or branched, saturated or mono- or polyunsaturated alkyl or alkenyl radical having from about 8 to about 24, preferably from about 12 to about 22, more preferably from about 16 to about 18 carbon atoms, n is 0 or from about 1 to about 12, more preferably from about 2 to about 4, and X is an alkali metal or alkaline earth metal ion or protonated triethanolamine or the ammonium ion,

straight-chain or branched, saturated or mono- or polyunsaturated alkylcarboxylic acids containing from about 8 to about 24, preferably from about 12 to about 22, more preferably from about 16 to about 18 carbon atoms,

straight-chain or branched, saturated or mono- or polyunsaturated alkyl phosphates containing from about 8 to about 24, preferably from about 12 to about 22, more preferably from about 16 to about 18 carbon atoms,

Alkyl isethionate whose alkyl group is selected from a branched or unbranched C₆ to C₂₂, preferably C₁₀ to C₁₈, more preferably C₁₂ to C₁₆ alkyl group, in particular sodium cocoyl isethionate,

Alkyl glycoside carboxylic acids whose alkyl group is selected from a branched or unbranched C₆ to C₂₂, preferably C₁₀ to C₁₈, more preferably C₁₂ to C₁₆ alkyl group,

Alkyl sulfosuccinates, the two alkyl groups of which are selected from identical or different, branched or unbranched C₂ to C₁₂, preferably C₄ to C₁₀, more preferably C₆ to C₈ alkyl groups,

Alkyl taurates, whose alkyl group is selected from a branched or unbranched C₆ to C₂₂, preferably C₁₀ to C₁₈, more preferably C₁₂ to C₁₆ alkyl group,

Alkyl sarcosinates, whose alkyl group is selected from a branched or unbranched C6 to C22, preferably C₁₀ to C₁₈, more preferably C₁₂ to C₁₆ alkyl group,

Sulfonates of unsaturated fatty acids with from about 8 to about 24, preferably from about 12 to about 22, more preferably from about 16 to about 18 C atoms and from about 1 to about 6 double bonds,

wherein the counterion of the anionic surfactant is an alkali or alkaline earth metal ion or a protonated triethanolamine or the ammonium ion.

Particularly preferred anionic surfactants are straight-chain or branched alkyl ether sulfates containing an alkyl radical with from about 8 to about 18 and in particular with from about 10 to about 16 carbon atoms and from about 1 to about 6 and in particular 2 to 4 ethylene oxide units. Very preferably, the surfactant mixture of anionic and amphoteric/zwitterionic surfactants contains sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO⁻ or —SO₃⁻ group in the molecule Amphoteric/zwitterionic surfactants also include surface-active compounds which, in addition to a C₈-C₂₄ alkyl or acyl group, contain at least one free amino group and at least one —COOH or —SO₃H group and are capable of forming internal salts.

According to a preferred embodiment of the present invention, the amphoteric surfactants in the cosmetic agent are selected from the group of

Alkyl betaine comprising at least one saturated or unsaturated, branched or unbranched C₆ to C₂₂, preferably C₁₀ to C₁₈, more preferably C₁₂ to C₁₆ alkyl group,

alkyl amphodiacetate or alkyl amphodiacetate comprising a saturated or unsaturated, branched or unbranched C₆ to C₂₂, preferably C₁₀ to C₁₈, more preferably C₁₂ to C₁₆ alkyl group, with an alkali or alkaline earth metal counterion, and

alkylamidopropyl betaine comprising at least one saturated or unsaturated, branched or unbranched C6 to C22, preferably C₁₀ to C18, more preferably C12 to C16 alkyl group.

Particularly suitable amphoteric/zwitterionic surfactants include those known under the INCI designation cocamidopropyl betaine and disodium cocoamphodiacetate.

According to a preferred embodiment of the present invention, the nonionic surfactant is selected from the group of

Alkyl glucamide comprising a saturated or unsaturated, branched or unbranched C₆ to C₂₂, preferably C₁₀ to C₁₈, more preferably C₁₂ to C₁₆ alkyl group,

An alkyl fructoside comprising a saturated or unsaturated, branched or unbranched C₆ to C₂₂, preferably C₁₀ to C₁₈, more preferably C₁₂ to C₁₆ alkyl group,

Alkylglucoside comprising a saturated or unsaturated, branched or unbranched C₆ to C₂₂, preferably C₁₀ to C₁₈, more preferably C₁₂ to C₁₆ alkyl group,

Alkyl alcohol alkoxylate of the formula R₁₀(OR₁₁)ₘOH, in which R₁₀ represents a linear or branched C₆ to C₂₂, preferably C₁₀ to C₁₈, more preferably C₁₂ to C₁₆ alkyl group, R₁₁ represents a C₂ to C₄, preferably a C₂ alkyl group, and ₘ represents from about 1 to about 10, preferably from about 2 to about 6, more preferably 2 to 6.

Alkyl esters of the formula R₁₂COOR₁₃, in which R₁₂ represents a linear or branched C6 to C22, preferably C₁₀ to C₁₈, more preferably C₁₂ to C₁₆ alkyl group, R₁₃ represents a C₁ to C₄, preferably a C₂ alkyl group.

According to a preferred embodiment of the present invention, the cosmetic agent contains two structurally different surfactants. It is particularly preferred that the cosmetic agent contains two structurally different surfactants from one another, preferably the cosmetic agent contains two structurally different cationic surfactants from one another, or the cosmetic agent contains a cationic surfactant and a nonionic surfactant.

The cosmetic compound may contain, in addition or as an alternative to a synthetic polymer, at least one natural or synthetic wax having a melting point above 37° C. as a firming compound.

Natural or synthetic waxes may be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable are the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate.

The wax components can also be selected from the group of esters of saturated, unbranched alkanecarboxylic acids having a chain length of from about 22 to about 44 carbon atoms and saturated, unbranched alcohols having a chain length of from about 22 to about 44 carbon atoms, provided that the wax component or the totality of wax components are solid at room temperature. Silicone waxes, for example stearyltrimethylsilane/stearyl alcohol, may also be beneficial.

Natural, chemically modified and synthetic waxes can be used alone or in combination. Thus, several waxes can also be used. Furthermore, a number of wax compounds, possibly mixed with other additives, are also commercially available. The products sold under the designations “Special Wax 7686 OE” (a mixture of cetyl palmitate, beeswax, microcrystalline wax and polyethylene with a melting range of 73-75° C.; manufacturer: Kahl & Co), Polywax® GP 200 (a mixture of stearyl alcohol and polyethylene glycol stearate with a melting point of 47-51° C.; manufacturer: Croda) and “Softceresin® FL 400” (a vaseline/vaseline oil/wax mixture with a melting point of 50-54° C.; manufacturer: Parafluid Mineral Oil Company) are examples of mixtures that can be used.

Preferably, the wax is selected from carnauba wax (INCI: Copernicia cerifera Cera) Beeswax (INCI: Beeswax), petrolatum (INCI), microcrystalline wax and especially mixtures thereof.

Preferred blends include the combination of carnauba wax (INCI: Copernicia cerifera Cera), petrolatum and microcrystalline wax or the combination of beeswax (INCI: Beeswax) and Petrolatum.

Wax or the wax components should be solid at 25° C. and should melt in the range>37° C.

The composition for treating a keratinous material contains a firming compound preferably in a total amount of about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic polymers, anionic polymers, cationic polymers, waxes, protein hydrolysates, amino acids, oligopeptides, vitamins, provitamins, vitamin precursors, betaines, biochinones, purine (derivatives), care substances, plant extracts, silicones, ester oils, UV light filters, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

In preferred embodiments 1 to 268, the preferred organic silicon compounds are combined with the preferred thickeners. The following combinations are present in cosmetic products in preferred embodiments of the present disclosure with other ingredients described above:

| | Silane compound | other ingredients |
|---|---|---|
| 1 | (3-Aminopropyl)trimethoxysilane | Xanthan |
| 2 | (3-Aminopropyl)triethoxysilane | Xanthan |
| 3 | (2-Aminoethyl)trimethoxysilane | Xanthan |
| 4 | (2-Aminoethyl)triethoxysilane | Xanthan |
| 5 | (3-Dimethylaminopropyl)trimethoxysilane | Xanthan |
| 6 | (3-Dimethylaminopropyl)triethoxysilane | Xanthan |
| 7 | (2-Dimethylaminoethyl)trimethoxysilane | Xanthan |
| 8 | (2-Dimethylaminoethyl)triethoxysilane | Xanthan |
| 9 | 3-(trimethoxysilyl)-N-[3- | Xanthan |
| | (trimethoxysilyl)propyl]-1-propanamine | |
| 10 | 3-(triethoxysilyl)-N-[3-(triethoxysilyl)propyl]- | Xanthan |
| | 1-propanamine | |
| 11 | N-methyl-3-(trimethoxysilyl)-N-[3- | Xanthan |
| | (trimethoxysilyl)propyl]-1-propanamine | |
| 12 | N-Methyl-3-(triethoxysilyl)-N-[3- | Xanthan |
| | (triethoxysilyl)propyl]-1-propanamine | |
| 13 | 2-[Bis[3-(trimethoxysilyl)propyl]amino]- | Xanthan |
| | ethanol | |
| 14 | 2-[bis[3-(triethoxysilyl)propyl]amino]ethanol | Xanthan |
| 15 | 3-(trimethoxysilyl)-N,N-bis[3- | Xanthan |
| | (trimethoxysilyl)propyl]-1-propanamine | |
| 16 | 3-(triethoxysilyl)-N,N-bis[3- | Xanthan |
| | (triethoxysilyl)propyl]-1-propanamine | |
| 17 | N1,N1-Bis[3-(trimethoxysilyl)propyl]-1,2- | Xanthan |
| | ethanediamine | |
| 18 | N1,N1-Bis[3-(triethoxysilyl)propyl]-1,2- | Xanthan |
| | ethanediamine | |
| 19 | N,N-Bis[3-(trimethoxysilyl)propyl]-2-propen- | Xanthan |
| | 1-amine | |
| 20 | N,N-Bis[3-(triethoxysilyl)propyl]-2-propen-1- | Xanthan |
| | amine | |
| 21 | Methyltrimethoxysilane | Xanthan |
| 22 | Methyltriethoxysilane | Xanthan |
| 23 | Ethyltrimethoxysilane | Xanthan |
| 24 | Ethyltriethoxysilane | Xanthan |
| 25 | Octyltrimethoxysilane | Xanthan |
| 26 | Octyltriethoxysilane | Xanthan |
| 27 | Dodecyltrimethoxysilane | Xanthan |
| 28 | Dodecyltriethoxysilane | Xanthan |
| 29 | (3-Aminopropyl)trimethoxysilane | Guar gum |
| 30 | (3-Aminopropyl)triethoxysilane | Guar gum |
| 31 | (2-Aminoethyl)trimethoxysilane | Guar gum |
| 32 | (2-Aminoethyl)triethoxysilane | Guar gum |
| 33 | (3-Dimethylaminopropyl)trimethoxysilane | Guar gum |
| 34 | (3-Dimethylaminopropyl)triethoxysilane | Guar gum |
| 35 | (2-Dimethylaminoethyl)trimethoxysilane | Guar gum |
| 36 | (2-Dimethylaminoethyl)triethoxysilane | Guar gum |
| 37 | 3-(trimethoxysilyl)-N-[3- | Guar gum |
| | (trimethoxysilyl)propyl]-1-propanamine | |
| 38 | 3-(triethoxysilyl)-N-[3-(triethoxysilyl)propyl]- | Guar gum |
| | 1-propanamine | |
| 39 | N-methyl-3-(trimethoxysilyl)-N-[3- | Guar gum |
| | (trimethoxysilyl)propyl]-1-propanamine | |
| 40 | N-Methyl-3-(triethoxysilyl)-N-[3- | Guar gum |
| | (triethoxysilyl)propyl]-1-propanamine | |
| 41 | 2-[Bis[3-(trimethoxysilyl)propyl]amino]- | Guar gum |
| | ethanol | |
| 42 | 2-[bis[3-(triethoxysilyl)propyl]amino]ethanol | Guar gum |
| 43 | 3-(trimethoxysilyl)-N,N-bis[3- | Guar gum |
| | (trimethoxysilyl)propyl]-1-propanamine | |
| 44 | 3-(triethoxysilyl)-N,N-bis[3- | Guar gum |
| | (triethoxysilyl)propyl]-1-propanamine | |
| 45 | N1,N1-Bis[3-(trimethoxysilyl)propyl]-1,2- | Guar gum |
| | ethanediamine | |
| 46 | N1,N1-Bis[3-(triethoxysilyl)propyl]-1,2- | Guar gum |
| | ethanediamine | |
| 47 | N,N-Bis[3-(trimethoxysilyl)propyl]-2-propen- | Guar gum |
| | 1-amine | |
| 48 | N,N-Bis[3-(triethoxysilyl)propyl]-2-propen-1- | Guar gum |
| | amine | |
| 49 | Methyltrimethoxysilane | Guar gum |
| 50 | Methyltriethoxysilane | Guar gum |
| 51 | Ethyltrimethoxysilane | Guar gum |
| 52 | Ethyltriethoxysilane | Guar gum |
| 53 | Octyltrimethoxysilane | Guar gum |
| 54 | Octyltriethoxysilane | Guar gum |
| 55 | Dodecyltrimethoxysilane | Guar gum |
| 56 | Dodecyltriethoxysilane | Guar gum |
| 57 | (3-Aminopropyl)trimethoxysilane | Microcrystalline cellulose |
| 58 | (3-Aminopropyl)triethoxysilane | Microcrystalline cellulose |
| 59 | (2-Aminoethyl)trimethoxysilane | Microcrystalline cellulose |
| 60 | (2-Aminoethyl)triethoxysilane | Microcrystalline cellulose |
| 61 | (3-Dimethylaminopropyl)trimethoxysilane | Microcrystalline cellulose |
| 62 | (3-Dimethylaminopropyl)triethoxysilane | Microcrystalline cellulose |
| 63 | (2-Dimethylaminoethyl)trimethoxysilane | Microcrystalline cellulose |
| 64 | (2-Dimethylaminoethyl)triethoxysilane | Microcrystalline cellulose |
| 65 | 3-(trimethoxysilyl)-N-[3- | Microcrystalline cellulose |
| | (trimethoxysilyl)propyl]-1-propanamine | |
| 66 | 3-(triethoxysilyl)-N-[3-(triethoxysilyl)propyl]- | Microcrystalline cellulose |
| | 1-propanamine | |
| 67 | N-methyl-3-(trimethoxysilyl)-N-[3- | Microcrystalline cellulose |
| | (trimethoxysilyl)propyl]-1-propanamine | |
| 68 | N-Methyl-3-(triethoxysilyl)-N-[3- | Microcrystalline cellulose |
| | (triethoxysilyl)propyl]-1-propanamine | |
| 69 | 2-[Bis[3-(trimethoxysilyl)propyl]amino]- | Microcrystalline cellulose |
| | ethanol | |
| 70 | 2-[bis[3-(triethoxysilyl)propyl]amino]ethanol | Microcrystalline cellulose |
| 71 | 3-(trimethoxysilyl)-N,N-bis[3- | Microcrystalline cellulose |
| | (trimethoxysilyl)propyl]-1-propanamine | |
| 72 | 3-(triethoxysilyl)-N,N-bis[3- | Microcrystalline cellulose |
| | (triethoxysilyl)propyl]-1-propanamine | |
| 73 | N1,N1-Bis[3-(trimethoxysilyl)propyl]-1,2- | Microcrystalline cellulose |
| | ethanediamine | |
| 74 | N1,N1-Bis[3-(triethoxysilyl)propyl]-1,2- | Microcrystalline cellulose |
| | ethanediamine | |
| 75 | N,N-Bis[3-(trimethoxysilyl)propyl]-2-propen- | Microcrystalline cellulose |
| | 1-amine | |
| 76 | N,N-Bis[3-(triethoxysilyl)propyl]-2-propen-1- | Microcrystalline cellulose |
| | amine | |
| 77 | Methyltrimethoxysilane | Microcrystalline cellulose |
| 78 | Methyltriethoxysilane | Microcrystalline cellulose |
| 79 | Ethyltrimethoxysilane | Microcrystalline cellulose |
| 80 | Ethyltriethoxysilane | Microcrystalline cellulose |
| 81 | Octyltrimethoxysilane | Microcrystalline cellulose |
| 82 | Octyltriethoxysilane | Microcrystalline cellulose |
| 83 | Dodecyltrimethoxysilane | Microcrystalline cellulose |
| 84 | Dodecyltriethoxysilane | Microcrystalline cellulose |
| 85 | (3-Aminopropyl)trimethoxysilane | Carrageenan |
| 86 | (3-Aminopropyl)triethoxysilane | Carrageenan |
| 87 | (2-Aminoethyl)trimethoxysilane | Carrageenan |
| 88 | (2-Aminoethyl)triethoxysilane | Carrageenan |
| 89 | (3-Dimethylaminopropyl)trimethoxysilane | Carrageenan |
| 90 | (3-Dimethylaminopropyl)triethoxysilane | Carrageenan |
| 91 | (2-Dimethylaminoethyl)trimethoxysilane | Carrageenan |
| 92 | (2-Dimethylaminoethyl)triethoxysilane | Carrageenan |
| 93 | 3-(trimethoxysilyl)-N-[3- | Carrageenan |
| | (trimethoxysilyl)propyl]-1-propanamine | |
| 94 | 3-(triethoxysilyl)-N-[3-(triethoxysilyl)propyl]- | Carrageenan |
| | 1-propanamine | |
| 95 | N-methyl-3-(trimethoxysilyl)-N-[3- | Carrageenan |
| | (trimethoxysilyl)propyl]-1-propanamine | |
| 96 | N-Methyl-3-(triethoxysilyl)-N-[3- | Carrageenan |
| | (triethoxysilyl)propyl]-1-propanamine | |
| 97 | 2-[Bis[3-(trimethoxysilyl)propyl]amino]- | Carrageenan |
| | ethanol | |
| 98 | 2-[bis[3-(triethoxysilyl)propyl]amino]ethanol | Carrageenan |
| 99 | 3-(trimethoxysilyl)-N,N-bis[3- | Carrageenan |
| | (trimethoxysilyl)propyl]-1-propanamine | |
| 100 | 3-(triethoxysilyl)-N,N-bis[3- | Carrageenan |
| | (triethoxysilyl)propyl]-1-propanamine | |
| 101 | N1,N1-Bis[3-(trimethoxysilyl)propyl]-1,2- | Carrageenan |
| | ethanediamine | |
| 102 | N1,N1-Bis[3-(triethoxysilyl)propyl]-1,2- | Carrageenan |
| | ethanediamine | |
| 103 | N,N-Bis[3-(trimethoxysilyl)propyl]-2-propen- | Carrageenan |
| | 1-amine | |
| 104 | N,N-Bis[3-(triethoxysilyl)propyl]-2-propen-1- | Carrageenan |
| | amine | |
| 105 | Methyltrimethoxysilane | Carrageenan |
| 106 | Methyltriethoxysilane | Carrageenan |
| 107 | Ethyltrimethoxysilane | Carrageenan |
| 108 | Ethyltriethoxysilane | Carrageenan |
| 109 | Octyltrimethoxysilane | Carrageenan |
| 110 | Octyltriethoxysilane | Carrageenan |
| 111 | Dodecyltrimethoxysilane | Carrageenan |
| 112 | Dodecyltriethoxysilane | Carrageenan |
| 113 | (3-Aminopropyl)trimethoxysilane | Locust bean gum |
| 114 | (3-Aminopropyl)triethoxysilane | Locust bean gum |
| 115 | (2-Aminoethyl)trimethoxysilane | Locust bean gum |
| 116 | (2-Aminoethyl)triethoxysilane | Locust bean gum |
| 117 | (3-Dimethylaminopropyl)trimethoxysilane | Locust bean gum |
| 118 | (3-Dimethylaminopropyl)triethoxysilane | Locust bean gum |
| 119 | (2-Dimethylaminoethyl)trimethoxysilane | Locust bean gum |
| 120 | (2-Dimethylaminoethyl)triethoxysilane | Locust bean gum |
| 121 | 3-(trimethoxysilyl)-N-[3- | Locust bean gum |
| | (trimethoxysilyl)propyl]-1-propanamine | |
| 122 | 3-(triethoxysilyl)-N-[3-(triethoxysilyl)propyl]- | Locust bean gum |
| | 1-propanamine | |
| 123 | N-methyl-3-(trimethoxysilyl)-N-[3- | Locust bean gum |
| | (trimethoxysilyl)propyl]-1-propanamine | |
| 124 | N-Methyl-3-(triethoxysilyl)-N-[3- | Locust bean gum |
| | (triethoxysilyl)propyl]-1-propanamine | |
| 125 | 2-[Bis[3-(trimethoxysilyl)propyl]amino]- | Locust bean gum |
| | ethanol | |
| 126 | 2-[bis[3-(triethoxysilyl)propyl]amino]ethanol | Locust bean gum |
| 127 | 3-(trimethoxysilyl)-N,N-bis[3- | Locust bean gum |
| | (trimethoxysilyl)propyl]-1-propanamine | |
| 128 | 3-(triethoxysilyl)-N,N-bis[3- | Locust bean gum |
| | (triethoxysilyl)propyl]-1-propanamine | |
| 129 | N1,N1-Bis[3-(trimethoxysilyl)propyl]-1,2- | Locust bean gum |
| | ethanediamine | |
| 130 | N1,N1-Bis[3-(triethoxysilyl)propyl]-1,2- | Locust bean gum |
| | ethanediamine | |
| 131 | N,N-Bis[3-(trimethoxysilyl)propyl]-2-propen- | Locust bean gum |
| | 1-amine | |
| 132 | N,N-Bis[3-(triethoxysilyl)propyl]-2-propen-1- | Locust bean gum |
| | amine | |
| 133 | Methyltrimethoxysilane | Locust bean gum |
| 134 | Methyltriethoxysilane | Locust bean gum |
| 135 | Ethyltrimethoxysilane | Locust bean gum |
| 136 | Ethyltriethoxysilane | Locust bean gum |
| 137 | Octyltrimethoxysilane | Locust bean gum |
| 138 | Octyltriethoxysilane | Locust bean gum |
| 139 | Dodecyltrimethoxysilane | Locust bean gum |
| 140 | Dodecyltriethoxysilane | Locust bean gum |
| 141 | Propyltrimethoxysilane | Xanthan |
| 142 | Propyltriethoxysilane | Xanthan |
| 143 | Hexyltrimethoxysilane | Xanthan |
| 144 | Hexyltriethoxysilane | Xanthan |
| 145 | Octadecyltrimethoxysilane | Xanthan |
| 146 | Octadecyltriethoxysilane | Xanthan |
| 147 | (3-Aminopropyl)triethoxysilane + | Xanthan |
| | Methyltrimethoxysilane | |
| 148 | (3-Aminopropyl)triethoxysilane + | Xanthan |
| | Methyltriethoxysilane | |
| 149 | (3-Aminopropyl)triethoxysilane + | Xanthan |
| | Ethyltrimethoxysilane | |
| 150 | (3-Aminopropyl)triethoxysilane + | Xanthan |
| | Ethyltriethoxysilane | |
| 151 | (3-Aminopropyl)triethoxysilane + | Xanthan |
| | Propyltrimethoxysilane | |
| 152 | (3-Aminopropyl)triethoxysilane + | Xanthan |
| | Propyltriethoxysilane | |
| 153 | (3-Aminopropyl)triethoxysilane + | Xanthan |
| | Hexyltrimethoxysilane | |
| 154 | (3-Aminopropyl)triethoxysilane + | Xanthan |
| | Hexyltriethoxysilane | |
| 155 | (3-Aminopropyl)triethoxysilane + | Xanthan |
| | Octyltrimethoxysilane | |
| 156 | (3-Aminopropyl)triethoxysilane + | Xanthan |
| | Octyltriethoxysilane | |
| 157 | (3-Aminopropyl)triethoxysilane + | Xanthan |
| | Dodecyltrimethoxysilane | |
| 158 | (3-Aminopropyl)triethoxysilane + | Xanthan |
| | Dodecyltriethoxysilane | |
| 159 | (3-aminopropyl)triethoxysilane + | Xanthan |
| | Octadecyltrimethoxysilane | |
| 160 | (3-aminopropyl)triethoxysilane + | Xanthan |
| | Octadecyltriethoxysilane | |
| 161 | Propyltrimethoxysilane | Guar gum |
| 162 | Propyltriethoxysilane | Guar gum |
| 163 | Hexyltrimethoxysilane | Guar gum |
| 164 | Hexyltriethoxysilane | Guar gum |
| 165 | Octadecyltrimethoxysilane | Guar gum |
| 166 | Octadecyltriethoxysilane | Guar gum |
| 167 | (3-Aminopropyl)triethoxysilane + | Guar gum |
| | Methyltrimethoxysilane | |
| 168 | (3-Aminopropyl)triethoxysilane + | Guar gum |
| | Methyltriethoxysilane | |
| 169 | (3-Aminopropyl)triethoxysilane + | Guar gum |
| | Ethyltrimethoxysilane | |
| 170 | (3-Aminopropyl)triethoxysilane + | Guar gum |
| | Ethyltriethoxysilane | |
| 171 | (3-Aminopropyl)triethoxysilane + | Guar gum |
| | Propyltrimethoxysilane | |
| 172 | (3-Aminopropyl)triethoxysilane + | Guar gum |
| | Propyltriethoxysilane | |
| 173 | (3-Aminopropyl)triethoxysilane + | Guar gum |
| | Hexyltrimethoxysilane | |
| 174 | (3-Aminopropyl)triethoxysilane + | Guar gum |
| | Hexyltriethoxysilane | |
| 175 | (3-Aminopropyl)triethoxysilane + | Guar gum |
| | Octyltrimethoxysilane | |
| 176 | (3-Aminopropyl)triethoxysilane + | Guar gum |
| | Octyltriethoxysilane | |
| 177 | (3-Aminopropyl)triethoxysilane + | Guar gum |
| | Dodecyltrimethoxysilane | |
| 178 | (3-Aminopropyl)triethoxysilane + | Guar gum |
| | Dodecyltriethoxysilane | |
| 179 | (3-Aminopropyl)triethoxysilane + | Guar gum |
| | octyldecyltrimethoxysilane | |
| 180 | (3-aminopropyl)triethoxysilane + | Guar gum |
| | Octadecyltriethoxysilane | |
| 181 | Propyltrimethoxysilane | Microcrystalline cellulose |
| 182 | Propyltriethoxysilane | Microcrystalline cellulose |
| 183 | Hexyltrimethoxysilane | Microcrystalline cellulose |
| 184 | Hexyltriethoxysilane | Microcrystalline cellulose |
| 185 | Octadecyltrimethoxysilane | Microcrystalline cellulose |
| 186 | Octadecyltriethoxysilane | Microcrystalline cellulose |
| 187 | (3-Aminopropyl)triethoxysilane + | Microcrystalline cellulose |
| | Methyltrimethoxysilane | |
| 188 | (3-Aminopropyl)triethoxysilane + | Microcrystalline cellulose |
| | Methyltriethoxysilane | |
| 189 | (3-Aminopropyl)triethoxysilane + | Microcrystalline cellulose |
| | Ethyltrimethoxysilane | |
| 190 | (3-Aminopropyl)triethoxysilane + | Microcrystalline cellulose |
| | Ethyltriethoxysilane | |
| 191 | (3-Aminopropyl)triethoxysilane + | Microcrystalline cellulose |
| | Propyltrimethoxysilane | |
| 192 | (3-Aminopropyl)triethoxysilane + | Microcrystalline cellulose |
| | Propyltriethoxysilane | |
| 193 | (3-Aminopropyl)triethoxysilane + | Microcrystalline cellulose |
| | Hexyltrimethoxysilane | |
| 194 | (3-Aminopropyl)triethoxysilane + | Microcrystalline cellulose |
| | Hexyltriethoxysilane | |
| 195 | (3-Aminopropyl)triethoxysilane + | Microcrystalline cellulose |
| | Octyltrimethoxysilane | |
| 196 | (3-Aminopropyl)triethoxysilane + | Microcrystalline cellulose |
| | Octyltriethoxysilane | |
| 197 | (3-Aminopropyl)triethoxysilane + | Microcrystalline cellulose |
| | Dodecyltrimethoxysilane | |
| 198 | (3-Aminopropyl)triethoxysilane + | Microcrystalline cellulose |
| | Dodecyltriethoxysilane | |
| 199 | (3-Aminopropyl)triethoxysilane + | Microcrystalline cellulose |
| | octyldecyltrimethoxysilane | |
| 200 | (3-Aminopropyl)triethoxysilane + | Microcrystalline cellulose |
| | Octyldecyltriethoxysilane | |
| 201 | Propyltrimethoxysilane | Carrageenan |
| 202 | Propyltriethoxysilane | Carrageenan |
| 203 | Hexyltrimethoxysilane | Carrageenan |
| 204 | Hexyltriethoxysilane | Carrageenan |
| 205 | Octadecyltrimethoxysilane | Carrageenan |
| 206 | Octadecyltriethoxysilane | Carrageenan |
| 207 | (3-Aminopropyl)triethoxysilane + | Carrageenan |
| | Methyltrimethoxysilane | |
| 208 | (3-Aminopropyl)triethoxysilane + | Carrageenan |
| | Methyltriethoxysilane | |
| 209 | (3-Aminopropyl)triethoxysilane + | Carrageenan |
| | Ethyltrimethoxysilane | |
| 210 | (3-Aminopropyl)triethoxysilane + | Carrageenan |
| | Ethyltriethoxysilane | |
| 211 | (3-Aminopropyl)triethoxysilane + | Carrageenan |
| | Propyltrimethoxysilane | |
| 212 | (3-Aminopropyl)triethoxysilane + | Carrageenan |
| | Propyltriethoxysilane | |
| 213 | (3-Aminopropyl)triethoxysilane + | Carrageenan |
| | Hexyltrimethoxysilane | |
| 214 | (3-Aminopropyl)triethoxysilane + | Carrageenan |
| | Hexyltriethoxysilane | |
| 215 | (3-Aminopropyl)triethoxysilane + | Carrageenan |
| | Octyltrimethoxysilane | |
| 216 | (3-Aminopropyl)triethoxysilane + | Carrageenan |
| | Octyltriethoxysilane | |
| 217 | (3-Aminopropyl)triethoxysilane + | Carrageenan |
| | Dodecyltrimethoxysilane | |
| 218 | (3-Aminopropyl)triethoxysilane + | Carrageenan |
| | Dodecyltriethoxysilane | |
| 219 | (3-aminopropyl)triethoxysilane + | Carrageenan |
| | Octadecyltrimethoxysilane | |
| 220 | (3-aminopropyl)triethoxysilane + | Carrageenan |
| | Octadecyltriethoxysilane | |
| 221 | Propyltrimethoxysilane | Locust bean gum |
| 222 | Propyltriethoxysilane | Locust bean gum |
| 223 | Hexyltrimethoxysilane | Locust bean gum |
| 224 | Hexyltriethoxysilane | Locust bean gum |
| 225 | Octadecyltrimethoxysilane | Locust bean gum |
| 226 | Octadecyltriethoxysilane | Locust bean gum |
| 227 | (3-Aminopropyl)triethoxysilane + | Locust bean gum |
| | Methyltrimethoxysilane | |
| 228 | (3-Aminopropyl)triethoxysilane + | Locust bean gum |
| | Methyltriethoxysilane | |
| 229 | (3-Aminopropyl)triethoxysilane + | Locust bean gum |
| | Ethyltrimethoxysilane | |
| 230 | (3-Aminopropyl)triethoxysilane + | Locust bean gum |
| | Ethyltriethoxysilane | |
| 231 | (3-Aminopropyl)triethoxysilane + | Locust bean gum |
| | Propyltrimethoxysilane | |
| 232 | (3-Aminopropyl)triethoxysilane + | Locust bean gum |
| | Propyltriethoxysilane | |
| 233 | (3-Aminopropyl)triethoxysilane + | Locust bean gum |
| | Hexyltrimethoxysilane | |
| 234 | (3-Aminopropyl)triethoxysilane + | Locust bean gum |
| | Hexyltriethoxysilane | |
| 235 | (3-Aminopropyl)triethoxysilane + | Locust bean gum |
| | Octyltrimethoxysilane | |
| 236 | (3-Aminopropyl)triethoxysilane + | Locust bean gum |
| | Octyltriethoxysilane | |
| 237 | (3-Aminopropyl)triethoxysilane + | Locust bean gum |
| | Dodecyltrimethoxysilane | |
| 238 | (3-Aminopropyl)triethoxysilane + | Locust bean gum |
| | Dodecyltriethoxysilane | |
| 239 | (3-Aminopropyl)triethoxysilane + | Locust bean gum |
| | Octadecyltrimethoxysilane | |
| 240 | (3-Aminopropyl)triethoxysilane + | Locust bean gum |
| | Octadecyltriethoxysilane | |
| 241 | Methyltrimethoxysilane | Alginic acid (salt) |
| 242 | Methyltriethoxysilane | Alginic acid (salt) |
| 243 | Ethyltrimethoxysilane | Alginic acid (salt) |
| 244 | Ethyltriethoxysilane | Alginic acid (salt) |
| 245 | Propyltrimethoxysilane | Alginic acid (salt) |
| 246 | Propyltriethoxysilane | Alginic acid (salt) |
| 247 | Hexyltrimethoxysilane | Alginic acid (salt) |
| 248 | Hexyltriethoxysilane | Alginic acid (salt) |
| 249 | Octyltrimethoxysilane | Alginic acid (salt) |
| 250 | Octyltriethoxysilane | Alginic acid (salt) |
| 251 | Dodecyltrimethoxysilane | Alginic acid (salt) |
| 252 | Dodecyltriethoxysilane | Alginic acid (salt) |
| 253 | Octadecyltrimethoxysilane | Alginic acid (salt) |
| 254 | Octadecyltriethoxysilane | Alginic acid (salt) |
| 255 | (3-Aminopropyl)triethoxysilane + | Alginic acid (salt) |
| | Methyltrimethoxysilane | |
| 256 | (3-Aminopropyl)triethoxysilane + | Alginic acid (salt) |
| | Methyltriethoxysilane | |
| 257 | (3-Aminopropyl)triethoxysilane + | Alginic acid (salt) |
| | Ethyltrimethoxysilane | |
| 258 | (3-Aminopropyl)triethoxysilane + | Alginic acid (salt) |
| | Ethyltriethoxysilane | |
| 259 | (3-Aminopropyl)triethoxysilane + | Alginic acid (salt) |
| | Propyltrimethoxysilane | |
| 260 | (3-Aminopropyl)triethoxysilane + | Alginic acid (salt) |
| | Propyltriethoxysilane | |
| 261 | (3-Aminopropyl)triethoxysilane + | Alginic acid (salt) |
| | Hexyltrimethoxysilane | |
| 262 | (3-Aminopropyl)triethoxysilane + | Alginic acid (salt) |
| | Hexyltriethoxysilane | |
| 263 | (3-Aminopropyl)triethoxysilane + | Alginic acid (salt) |
| | Octyltrimethoxysilane | |
| 264 | (3-Aminopropyl)triethoxysilane + | Alginic acid (salt) |
| | Octyltriethoxysilane | |
| 265 | (3-Aminopropyl)triethoxysilane + | Alginic acid (salt) |
| | Dodecyltrimethoxysilane | |
| 266 | (3-Aminopropyl)triethoxysilane + | Alginic acid (salt) |
| | Dodecyltriethoxysilane | |
| 267 | (3-Aminopropyl)triethoxysilane + | Alginic acid (salt) |
| | Octadecyltrimethoxysilane | |
| 268 | (3-Aminopropyl)triethoxysilane + | Alginic acid (salt) |
| | Octadecyltriethoxysilane | |

The active ingredient combination of at least one organic silicon compound and the thickener may already be present in the agent for treating a keratinous material. In this embodiment, the agent for treating a keratinous material is already distributed in a ready-to-use form. In order to provide a formulation that is as stable as possible during storage, the agent itself is preferably packaged with low or no water.

Alternatively, at least one organic silicon compound is added a maximum of 12 hours, preferably a maximum of 6 hours, more preferably a maximum of 3 hours, even more preferably a maximum of 1 hour prior to application of the keratinous material treatment agent to a base comprising all of the ingredients of the keratinous material treatment agent except the at least one organic silicon compound.

Furthermore, alternatively, the organic silicon compound and the thickener are added to a cosmetic product only shortly before use, i.e. from about 1 minute to about 12 hours, preferably from about 2 minutes to about 6 hours, particularly preferably from about 1 minute to about 3 hours, especially preferably from about 1 minute to about 1 hour.

Another object of the present application is the use of a cosmetic composition as contemplated herein for treating a keratinous material,

for the shaping of keratinous material and/or
for the care of keratinous material.

With regard to further preferred embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the treatment of a keratinous material, comprising
a) at least one organic silicon compound and
b) at least one thickener.

2. A cosmetic composition for treating a keratinous material according to claim 1,
wherein
the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group,
R₃, R₄ independently represent a methyl group or an ethyl group,
a stands for the number 3 and
b stands for the number 0, and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d}″(OR₅″)_{c}″ (III),
c stands for an integer from about 1 to about 3,
d stands for the integer 3−c′,
c′ stands for an integer from about 1 to about 3,
d′ stands for the integer 3−c′,
c″ stands for an integer from about 1 to about 3,
d″ stands for the integer 3−c″,
e stands for 0 or 1,
f stands for 0 or 1,
g stands for 0 or 1,
h stands for 0 or 1,
with the proviso that at least one of the components of e, f, g and h is different from 0.

3. A cosmetic composition for treating a keratinous material according to claim 2,
wherein
the agent for treating a keratinous material comprises at least one organic silicon compound of formula (I) selected from the group of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane and/or
(2-Dimethylaminoethyl)triethoxysilane,
or wherein,
the agent for treating a keratinous material comprises at least one organic silicon compound of formula (II) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

4. A cosmetic agent for treating a keratinous material according to claim 1, wherein the organic silicon compound of formula (I) is present in the cosmetic agent in an amount of from about 0.01 to about 10% by weight and/or wherein the organic silicon compound of formula (II) is present in an amount of from about 0.01 to about 10% by weight,
and/or wherein the organic silicon compound of formula (I) is (3-aminopropyl)triethoxysilane and/or the organic silicon compound of formula (II) is 3-(triethoxysilyl)-N-[3-(triethoxysilyl)propyl]-1-propanamine.

5. A cosmetic agent for treating a keratinous material according to claim 1, wherein the agent for treating a keratinous material comprises at least one organic silicon compound of formula (IV),
R₉Si(OR₁₀)ₖ(R₁₁)ₘ (IV),
which is selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane
Octadecyltrimethoxysilane and/or
Octadecyltriethoxysilane

6. A cosmetic composition for treating a keratinous material according to claim 1, wherein the thickener comprises a crosslinked polymer, a polyelectrolyte or a crosslinked polyelectrolyte, preferably wherein the thickener is selected from the group of hydroxyethylcellulose, carboxymethylcelluloses, sodium carboxymethylcellulose, hydroxypropylmethylcellulose, hydroxypropylcellulose, ethylhydroxyethylcellulose, ethylcellulose, methylethylcellulose, Methyl cellulose, Cellulose powder, Microcrystalline cellulose, Polyvinyl alcohols, Polyacrylic acid and polymethacrylic acids, Polyacrylamides, Polyvinyl pyrrolidone, Polyethylene glycols, Alginic acid, Sodium alginate, Potassium alginate, Ammonium alginate, Calcium alginate, Propylene glycol alginate, agar-agar, carrageenan, furcellaran, locust bean gum, guar gum, tragacanth, gum arabic, xanthan, chitosan, cationized honey, karaya, Indian tragacanth, tara gum, locust bean gum, gellan, pectin and amidated pectin.

7. Cosmetic agent for treating a keratinous material according to claim 1, wherein the thickener comprises alginic acid and/or a salt of alginic acid.

8. A cosmetic agent for treating a keratinous material according to claim 1, wherein the thickener is present in the cosmetic agent in an amount of from about 0.001 to about 5% by weight.

9. Cosmetic agent for treating a keratinous material according to claim 1, wherein the cosmetic agent comprises an amphoteric and/or nonionic surfactant.

10. Cosmetic agent for treating a keratinous material according to claim 1, wherein the cosmetic agent for treating a keratinous material comprises at least two organic silicon compounds which are structurally different from one another.

11. A cosmetic agent for treating a keratinous material according to claim 1, wherein the agent for treating a keratinous material comprises—based on the total weight of the agent for treating a keratinous material:
from about 0.5 to about 3 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7 wt. % of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

12. (canceled)

13. Cosmetic agent for treating a keratinous material according to claim 2, wherein
the agent for treating a keratinous material comprises at least one organic silicon compound of formula (II) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

14. Cosmetic product for the treatment of a keratinous material as claimed in claim 2, wherein the organic silicon compound of the formula (I) is present in a quantity of from about 0.1 to about 4% by weight, based on the total weight of the cosmetic product.

15. Cosmetic product for the treatment of a keratinous material as claimed in claim 2, wherein the organic silicon compound of the formula (II), is present in a quantity of from about 0.1 to 6% by weight, based on the total weight of the cosmetic product.

16. Cosmetic product for the treatment of a keratinous material as claimed in claim 2, wherein the organic silicon compound of formula (I) is (3-aminopropyl)triethoxysilane and/or the organic silicon compound of formula (II) is 3-(triethoxysilyl)-N-[3-(triethoxysilyl) propyl]-1-propane amine.

17. A cosmetic agent for treating a keratinous material according to claim 1, wherein the thickener is present in the cosmetic agent in an amount of from about 0.05 to about 0.5% by weight, based on the total weight of the cosmetic agent.

18. Cosmetic agent for treating a keratinous material according to claim 9, wherein the cosmetic agent comprises an amphoteric and/or nonionic surfactant selected from the group of
alkyl betaine comprising at least one saturated or unsaturated, branched or unbranched C₆ to C₂₂,
alkyl amphodiacetate or alkyl amphodiacetate comprising a saturated or unsaturated, branched or unbranched C₆ to C₂₂, with an alkali or alkaline earth metal counterion, and
alkylamidopropyl betaine comprising at least one saturated or unsaturated, branched or unbranched C₆ to C₂₂,
and/or wherein the nonionic surfactant is selected from the group of
Alkyl glucamide comprising a saturated or unsaturated, branched or unbranched C₆ to C₂₂,
alkylglucosid comprising a saturated or unsaturated, branched or unbranched C₆ to C₂₂,
alkyl glucoside comprising a saturated or unsaturated, branched or unbranched C₆ to C₂₂, and
alkyl alcohol alkoxylate of the formula R₁₀(OR₁₁)ₘOH, in which R₁₀ represents a linear or branched C₆ to C₂₂, and ₘ represents from about 1 to about 10.
alkyl esters of the formula R₁₂COOR₁₃, in which R₁₂ represents a linear or branched C₆ to C₂₂, R₁₃ represents a C₁ to C₄.
